(19) United States
(12) Patent Application Publication
(10) Pub. No.: US 2015/0216742 A1
(43) Pub. Date: Aug. 6, 2015

(54) Absorbent Pad

(71) Applicant: Principle Business Enterprises, Inc., Dunbridge, OH (US)

(72) Inventors: Erin L. Johnson, Perrysburg, OH (US); Andrew J. Szypka, Curtice, OH (US)

(21) Appl. No.: 14/612,606

(22) Filed: Feb. 3, 2015

Related U.S. Application Data
(60) Provisional application No. 61/934,960, filed on Feb. 3, 2014.

(51) Int. Cl. A61F 13/537; A61F 13/539; A61F 13/51; A61F 13/84

(52) U.S. Cl. CPC A61F 13/5376; A61F 13/8405; A61F 13/539; A61F 13/52; A61F 2013/8408; A61F 2013/8414; A61F 2013/15373

(57) ABSTRACT

A disposable, absorbent “moisture management” pad for use to absorb fluids secreted or discharged from a user. The pads will protect the skin area from exudates/drainage by absorbing large amounts of fluid and locking it into a super absorbent core that remains dry on the outer surface next to the skin. The pad contains an ultra-thin core containing super absorbent polymer and two layers of tissue substrates, a water impervious non-woven back sheet and a water pervious non-woven top sheet.

### CROSS REFERENCE TO RELATED APPLICATIONS

This application claims the benefit of U.S. Provisional Patent Application No. 61/934,960 filed on Feb. 3, 2014.

### BACKGROUND OF THE INVENTION

The present invention relates to a disposable absorbent moisture management pad of the kind which is intended for daily use. In particular, the moisture absorbent pad is designed for absorbing fluids that are secreted or discharged from a user. One particular application for this pad is a nursing pad that can be positioned in the clothing of a nursing mother adjacent to the breast to manage discharges or secretions from the breast.

There are many prior art absorbent pads that have been utilized for applications such as nursing pads. These prior art pads have all had limitations that have limited the successful use of such pads. One of the primary disadvantages of the prior art pads are that they are very thick to include enough absorbent material to effectively manage the discharge or secretions that are created from the user. The thick bulky pads are not comfortable, do not conveniently fit within the clothing of the user and provide an unacceptable appearance. The prior art pads primarily rely upon a fibrous absorbent product known as fluff to absorb the fluids from the user. To have sufficient absorbent capacity there must be a significant quantity of fluff and this results in a very thick and unacceptable pad product. The absorbency to thickness ratio for such prior art pads is not good and results in a thick pad if there is sufficient absorbency capacity. The prior art pads, at least partially because of their thickness, must be made with a shape or contour that fits the anatomy of the user. The preformed shape of the pad results in a pad that takes up more space in packaging and in storage by the user, and these space issues limit the acceptability of these pads. The fluff product absorbs the fluid but does not act to contain or isolate the fluids from the surface of the pad that is in contact with the user. Accordingly, there is usually a layer of moisture containing fluff that is retained and positioned adjacent the user of the pad. The moisture in this layer of fluff holds the moisture adjacent the user and creates a wet environment adjacent to the user that is not beneficial in creating a dry condition adjacent the skin of the user. This problem is further exaserbated by the fact that the moisture contained in the fluff fibers can move back towards the user if pressure is applied to the fluff fibers. Pressure can be applied to the fluff fibers by the clothing of the user or by pressure applied to the outside of the pad that will result in the moisture being directed towards the user. This rewetting of the surface of the pad that is adjacent the user is an unacceptable feature of these prior art pads that results in a less than healthy environment for the skin of the user that is in the area adjacent the pad.

The prior art pads also do not control the discharge or secretions from the user in a way that manages any odor producing fluids. In particular, a nursing mother will have leakage or discharge from the breast that produce a milk or sour milk type of odor that can be unpleasant and somewhat embarrassing for the nursing mother. The prior art pads retain the fluids from the nursing mother but do not effectively manage the odor producing the components of these fluids. This is again a significant short coming of these prior art moisture control pads.

Accordingly, there is a need in the absorbent pad market for a pad that is significantly thinner than the prior art pads so that it is comfortable, fits within the users clothing and esthetically acceptable. The thin pad must have the ability to absorb a sufficient quantity of fluid to effectively manage the discharge or secretion from the user. There is a need for a pad that has a flat shape for ease of packing and storage, but that is sufficiently compliant that the pad will readily conform to the shape of the area of the user where the pad is positioned on the user. There is also a need for a moisture control pad that affectively encapsulates the discharges or discretions from the user in a way that effectively manages any odor producing characteristics of such fluids. There is also a need in the absorbent pad market for a pad that will maintain a dry surface on the skin of the user that is adjacent the pad. Such a pad will allow the skin of the user to remain healthy and the wearer will see a decrease in unhealthy skin issue relating to moisture accumulation. The moisture management pad of the present invention overcomes the deficiencies of the prior art pads.

Other objects and advantages of the present invention will become apparent to those skilled in the art upon a review of the following detailed description of the preferred embodiments and the accompanying drawings.

### SUMMARY OF THE INVENTION

The invention is directed to a pad that can be used to absorb fluids. The pad can be effectively used as a nursing pad. When used as a nursing pad, the pad is designed to collect fluids from the breasts. The pad utilizes a super absorbent polymer to retain the fluids from the user and to produce a pad that is very thin in size but capable of retaining a sufficient quantity of fluid. The pad has a first layer of non-woven material positioned on the first side of the core and a second layer of non-woven material positioned on the second side of the core. The first layer of non-woven material is designed to be positioned against the user and functions to wick fluids from the user into the core. The features of the invention will be more readily understood by referring to the attached drawings in connection with the following description. Various materials can be substituted for the materials listed as long as these materials perform the required criteria needed for the material.

### BRIEF DESCRIPTION OF THE DRAWINGS

FIG. 1 is a front elevational view of the pad of the present invention.

FIG. 2 is an exploded side elevational view of the pad.

FIG. 3 is a side cross sectional view of the pad.

FIG. 4 is a front elevational view of the pad.

FIG. 5A and B are side elevational views of the pad of FIG. 4.

FIG. 6 is a partial side elevational view of the pad being used.

FIG. 7. Is a partial cross sectional view of a portion of the pad.

### DETAILED DESCRIPTION OF THE PREFERRED EMBODIMENT(S)

The invention is directed to a pad that can be used to absorb fluids. More particularly the pad is designed to be used as a nursing pad. The nursing pad is designed to collect fluids from the breasts. The pad utilizes a super absorbent polymer to retain the fluids from the user and to produce a pad that is very thin in size but capable of retaining a sufficient quantity of fluid. The pad has a first layer of non-woven material positioned on the first side of the core and a second layer of non-woven material positioned on the second side of the core. The first layer of non-woven material is designed to be positioned against the user and functions to wick fluids from the user into the core. The features of the invention will be more readily understood by referring to the attached drawings in connection with the following description.

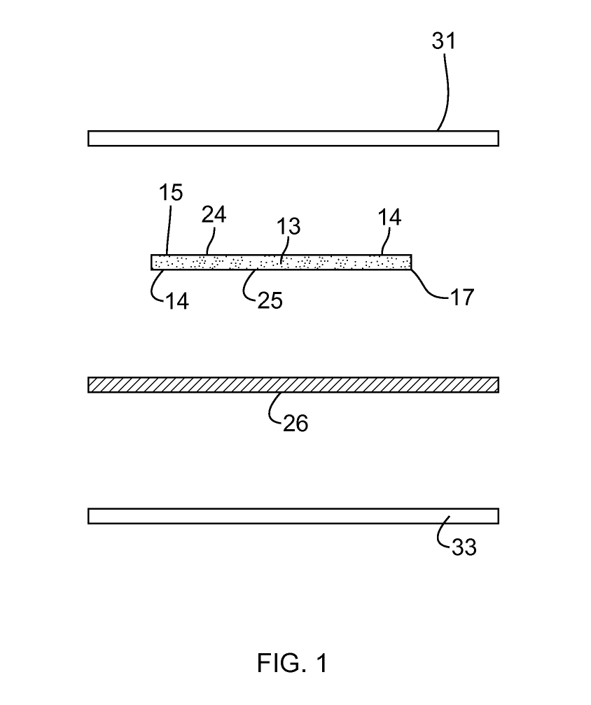

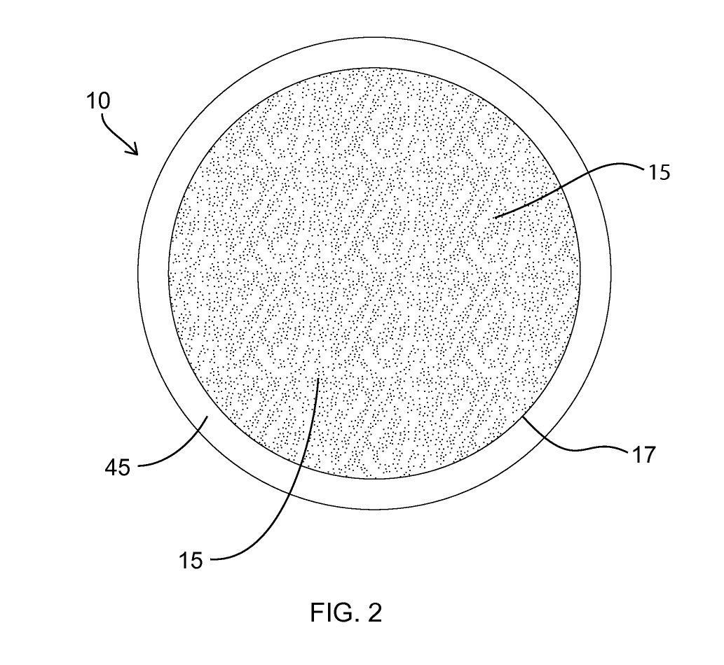

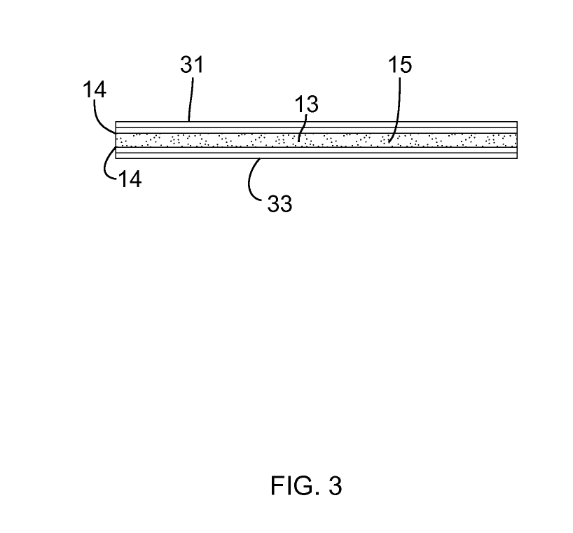

The features of the pad 10 are shown in FIGS. 1, 2 and 3. The pad has a core 15 that is a laminate of a super absorbent polymer and tissue. Gelok laminate material, such as, 5040-104 or 9040-104 are an example of a laminate that works particularly well for the core. The core has a super absorbent polymer powder 13 that is positioned between two layers of cellulosic tissue 14. It should be appreciated that outer materials, such as airlay composites and non-woven webs constructed with rayon cellulose acetate, polypropylene, polyester and bi-co fibers, can be used for the tissue layer 14. The non-woven webs can be spunbond, SMS, thru air or bi-co types of non-wovens. This paper like super absorbent core 15 has high fluid absorbency, but remains dry to the touch. The absorbent core 15 has a basis weight from about 50 gsm to about 250 gsm. The super absorbent laminate of the core is very effective at absorbing fluids and controlling or reducing odor. The core 15 has an absorbency from about 15 grams of fluid per pad (g/pad) to about 90 g/pad or 15 grams of fluid per mm of thickness (g/mm) to about 180 g/mm, with a preferred range of from about 20 g/pad to about 60 g/pad or from about 20 g/mm to about 90 g/mm. The fluid used to establish these results was a saline solution of water with a 0.9% salt level. This high absorbency ratio allows the core to absorb sufficient fluid while allowing the core to be acceptably thin. The core has a thickness from about 0.4 mm to about 1.5 mm with a preferred range of from about 0.5 mm to about 1 mm. The thinness of the core results in a pad 10 that has high conformability that does not require that the pad be conformed or molded to conform to the shape of the area where the pad is utilized. The high percentage of super absorbent polymer in the core, from about 30% to about 70% of the total basis weight of the pad, functions to effectively lock any fluids in the core area of the pad. The fluid remains in the core, absorbed in the super absorbent polymer, and does not flow out of the pad during standard use or when changing of the pad. This feature of the core helps to reduce or prevent the fluid from rewetting the first layer of material that is disposed adjacent the user. This results in reduced moisture at the pad skin interface which supports healthy dry skin in the area where the pad is used. The super absorbent material encapsulates or encloses the fluids from the user, including the odor producing components of the fluid. The super absorbent polymer physically and chemically bonds to the fluids and retains the fluids in the super absorbent polymer. The fluids and super absorbent polymer turn into a gel as the fluids are absorbed. The gel acts to keep the fluids from deteriorating and producing odors. Thus, the odors from the fluids are primarily retained in the super absorbent laminate and less odor producing components escape to the air adjacent the user. The quantity of super absorbent polymer in the core 15 can be adjusted to create a pad 10 with different levels of absorbency to accommodate the need of various users and various use conditions. The core 15 has an outer perimeter 17 that generally defines the outer limits of the core 15. The core 15 as shown in FIG. 2 is depicted as being substantially circular in shape. It should be appreciated, however, that other shapes such as squares, rectangles and other geometric configurations can be used for the core 15.

As shown in FIG. 2 a first layer 31 of non-woven material is positioned on the first side 24 of the core 15. A second layer 33 of non-woven material is positioned adjacent the second side 25 of the core 15. The first and second layer are preferably a spun bond-melt blown-spun bond polypropylene (SMS) or a thermal bond polypropylene non-woven material having a thickness from about 0.05 mm to about 0.25 mm and a basis weight from about 5 gsm to about 25 gsm. The first and second layers of nonwoven material are so thin that these layers have essentially no impact in the thickness of the pad. The thickness of the core 15 effectively determines the thickness of the pad 10. Other materials such as a cloth like film can be used for the second layer. The cloth like film can be a liquid impervious plastic material and it is usually preferred that this film be breathable to have better moisture control for the pad 10. The second layer 33 can also have a surface that faces away from the core that is printable.

A layer 26 of a dual coat transfer adhesive or a spray adhesive can be positioned on the second side 25 of the core 15. The layer 26 of adhesive is used to assist in securing the second layer 33 of non-woven material or cloth like film to the second side of the core 15.

A seal 45 is formed between the first and second layers of non-woven material. The seal can be made by heat sealing, ultrasonic bonding or by an adhesive. If a layer 26 of a dual coat adhesive or a spray adhesive is utilized, the outer perimeter of the layer can be used to form the seal between the first and second layers. The seal must be sufficiently strong to retain its integrity as the super absorbent core expands when it absorbs fluids. The seal 45 is positioned adjacent the outer boundary of the first and second layers 31, 33 of non-woven material. The seal 45 is formed so that the entire core 15 is positioned inside the confines of the seal 45. The area of the seal 45, on the outer periphery of the pad 10 provides an area that can be gripped by the user to position or remove the pad.

The first layer 31 of non-woven material is designed to wick fluid from the user, to the super absorbent laminate material 13 in the core 15. The first and second layers are usually a hydrophilic non-woven material as this works very well as a wicking layer. The second layer 33 of non-woven material can also be designed to function to wick moisture or fluids away from the user or the user's apparel to the super absorbent material in the core 15. It is also possible that the second layer 33 of non-woven material can be fluid impervious material that is designed to act as a fluid barrier and to retain fluids from the user in the core 15. The material of the second layer 33 can be a hydrophobic material that resists fluids and is generally impervious to the flow of fluids. It is preferable that the impervious second layer 33 be breathable to provide as comfortable an environment as possible for the user.

The seal 45 is designed to retain fluids from the user in the core 15. The super absorbent polymer contained in the core is designed to absorb and retain large quantities of fluids. Accordingly, the pad 10 will be capable of wicking fluids away from the user into the core 15 of the pad 10 where the fluids will be retained in the super absorbent polymer. The super absorbent laminate of the core is very effective at controlling or reducing odor. The high percentage of super absorbent polymer in the core functions to contain odors in the core area of the pad. It is also possible that an odor control substance can be incorporated into the material of the core to control odors from the fluids from the patient. Odor control agents such as baking soda, activated carbon and other known agents can be dispersed in the super absorbent polymer of the core. The absorption qualities of the super absorbent polymer contained in the core 15 allow the pad 10 to be thin, but at the same time manage a significantly large quantity of fluids that discharge, for example, from the breast of a nursing mother. The core 15 manages the fluids from the user by absorbing and retaining the fluids. This feature of the core reduces breakdown of the skin adjacent the pad, reduces odor issues and reduces the frequency that pads need to be changed. The thinness of the pad 10 provides a more comfortable and anesthetically pleasing pad. In practice it has been found that the thickness of the pad can be less then 30 mils and still function to manage fluids associated with a nursing mother. The pad is usually from about 15 mils to about 50 mils in thickness. The prior art absorbent pads traditionally have a thickness of more than 100 mils. The thinness of the pad 10 allows the pad to readily conform to the area of the user where the pad is used. It is not necessary to preshape or mold a shape into the pad to obtain acceptable conformance with the anatomy of the user. The pad has a bending length stiffness from about 40 mm to about 70 mm, using the INDA test method for non-woven materials, and this allows the pad to conform to the shape of the user. As an example, if the pad is used by a nursing mother, the pad will conform to the shape of the mother's breast and clothing without preshaping of the pad. This allows the pad to be packaged in a flat configuration which requires much less packaging and storage space for the pads 10.

The typical overall dimensions of the pad are from about 3.5 to about 5.5 inches in diameter. This provides a pad that will provide coverage for most moisture control applications.

The other key feature of this unique design is the pad's ultra-thin core that is made with super absorbent powder and cellulosic tissues. This material is a super absorbent laminate made by Gelok International. This paper like, super absorbent core allows for high fluid absorbency with an ultra-dry sensation on the exterior of the core. Accordingly, the pad feels much drier when contacted by a user. The pad does not rewet when handled. The pad can also be sterilized if desired.

In operation the pad 10 is positioned adjacent the user to collect fluids that leak or are secreted from the body. It is very important that the fluids that leak or secreted from the body are managed to maintain as healthy a condition as possible. The pad of this invention has a conformability from the component layers that assist in positioning the pad on the user or in the clothing of the user. The conformability of the pad helps the pad to assume the shape of the anatomy of the user when in use. The conformability of the pad also allows the pad to retain its shape and position relative to the user as the pad absorbs fluids from the user. The first layer 31 of non-woven material will come into contact with any fluids and wick the fluids to the super absorbent polymer in the core 15. The super absorbent polymer will absorb and retain the fluids within the core 15. The super absorbent laminate of the core is very effective at controlling or reducing odor. The high percentage of super absorbent polymer in the core functions to contain odors in the core area of the pad. The core 15 can absorb and retain, in the super absorbent polymer, as much as 3 to 6 times the quantity of fluids that can be just absorbed by the currently used absorbent fluff type of products. The pad 10 can also be worn or used for 6-10 hours due to the high absorptive properties of the super absorbent polymer material and this is considerably longer, 2-4 times, than the pads currently used. As the fluids are moved away from the user, the skin of the user will be kept dry and as healthy as possible. The second layer 33 of non-woven material can also be a fluid impervious material that will function to prevent any fluid in the core 15 from leaving the pad 10 through the second layer 33. It is desirable, however, that the first and second layers of non-woven material be breathable to provide good air flow around the skin where the pad is positioned. The breathable good air flow characteristics of the first and second layer further provide a healthy environment for the skin in the area adjacent to the pad 10. An antimicrobial or antifungal agent can be incorporated into the first layer 31 of non-woven material to provide additional protection for the skin where the pad is positioned. An antimicrobial and antifungal compound sold under the name AEG15 has been found to be very effective for this component of the pad.

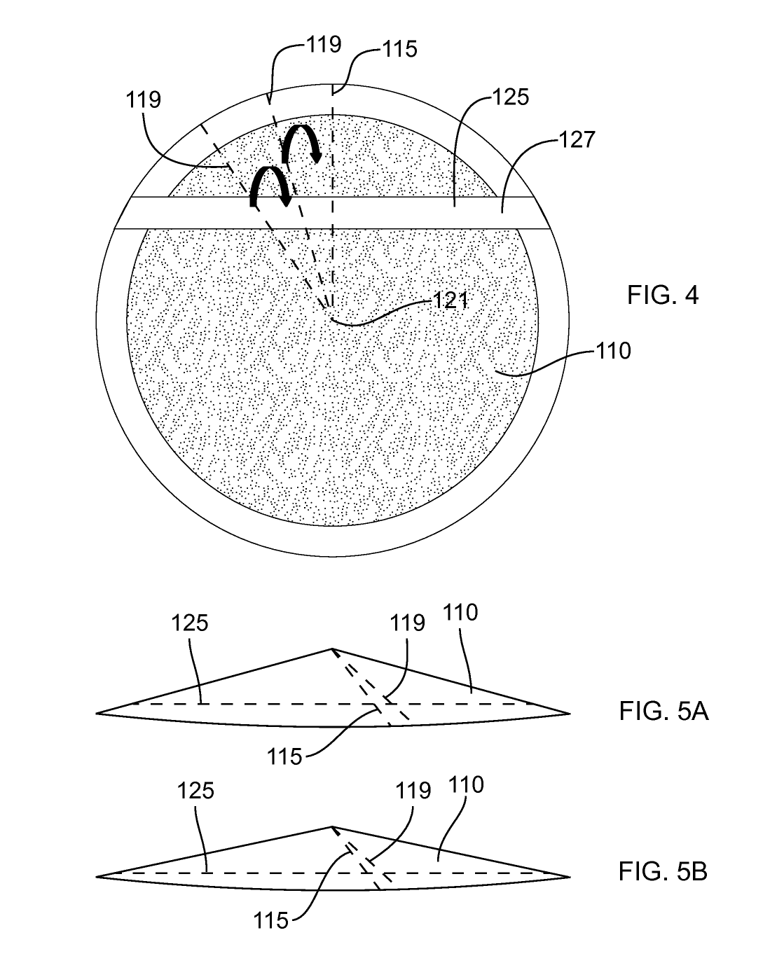

FIGS. 4-6 shows another feature of the pad that can be used to improve the useability of the pad. The pad 110 is constructed in the same way as the previously constructed pad 10, using same materials. As shown in FIGS. 4-6 the pad 110 has line 115 printed or embossed on the pad. Adjacent the line 115 are at least one fold line 119 positioned adjacent the line 115. The line 115 and the at least one fold line 119 can extend from the same point 121 on the pad 110 and extend outwardly in a radial direction to the outer periphery of the pad. In practice, it has been found desirable to have several fold lines 119 and the fold lines can be printed or embossed on the pad. The line 115 and the fold line 119 can be placed on either or both sides of the pad. In practice it has been found preferable to place the line and the fold lines on the side of the pad that is against the user when the pad is being used. An adhesive strip 125 is positioned on the pad to extend over the line 115 and at least on fold line 119. The adhesive strip is secured to the pad 110 on one side and has a release film 127 that covers and adhesive coating on the side of the adhesive strip that is spaced apart from the pad 110.

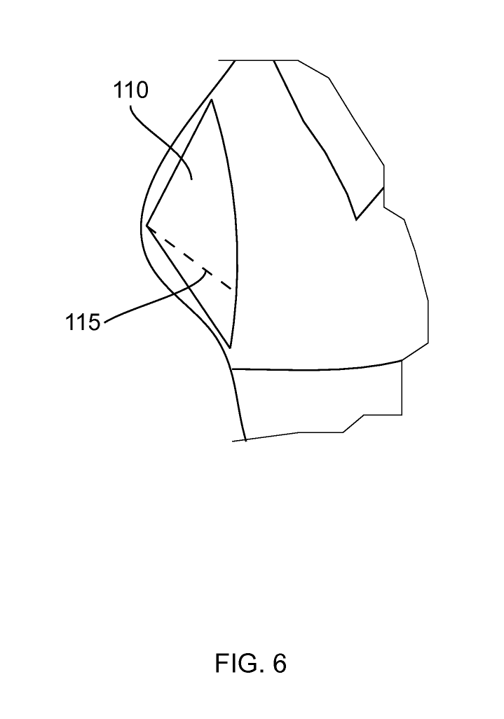

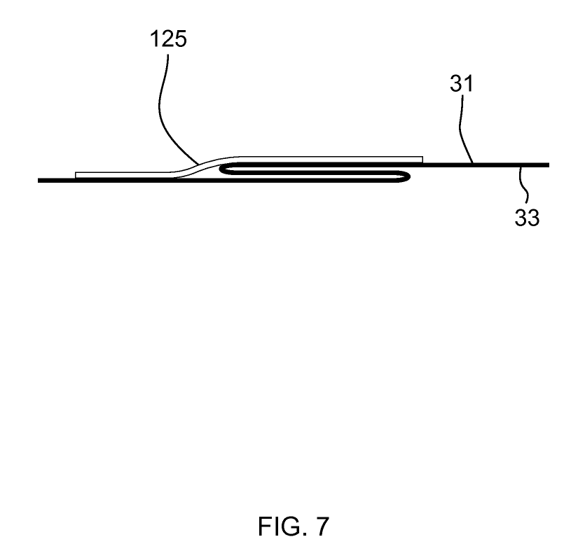

Although the pad 110 is readily conformable to the area of the user, there are some situations where it may be desirable to pre-shape the pad prior to use. The pad would still be stored in the flat configuration to facilitate shipping, storage, packaging and use. But once the pad 110 was removed from the packaging, it could be folded along line 115 which presents an ideal place to fold the pad to add contour to the pad. One of the fold lines 119 can be used as a guide to determine how much folding will take place in the pad 110 and the degree of shape or contour that is placed in the pad. Once the desired shape is produced in the pad, release film can be removed from the adhesive strip 125 and the adhesive strip used to secure the pad in the folded configuration. Once a desired shape has been established for the pad 110, the same fold line 119 can be used to achieve the desired configuration on a repeatable basis. The adhesive strip 125 can be made sufficiently long to secure the fold in the pad or the adhesive strip can be made longer and function to secure the pad 110 in the garment of a user as shown in FIG. 6.

The above detailed description of the present invention is given for explanatory purposes. It will be apparent to those skilled in the art that numerous changes and modifications can be made without departing from the scope of the invention. Accordingly, the whole of the foregoing description is to be construed in an illustrative and not a limitative sense, the scope of the invention being defined solely by the appended claims.

I claim:

1. An absorbent pad comprising:
a core of super absorbent polymer;
a first layer of non-woven material positioned on the first side of the core;
a second layer of non-woven material positioned on the second side of the core, the first and second layers having an outer peripheral boundary, the outer peripheral boundary extending beyond the outer peripheral edge of the core; and
a seal formed between the first and second layers of non-woven material, adjacent the outer boundary of the first and second layers, the pad having a thickness of less than 1.5 mm and an absorbency of at least 15 g/mm.

2. The pad of claim 1 wherein the super absorbent polymer of the core is suspended in a cellulose layer designed to wick fluids to the super absorbent layer.

3. The pad of claim 1 wherein the first layer of non-woven material is designed to wick fluids from the user to the super absorbent material.

4. The pad of claim 7 wherein the second layer of non-woven material is designed to wick fluids from the user to the core.

5. The pad of claim 7 wherein the second layer of non-woven material is a fluid impervious material that is designed to retain fluids from the user in the core.

6. The pad of claim 1 wherein the seal is designed to retain fluids from the user in the core.

7. The pad of claim 9 wherein the second layer is hydrophobic and breathable.

8. The pad of claim 1 wherein the pad has a thickness of from about 0.5 mm to about 1 mm.

9. The pad of claim 1 wherein an odor controls substance is dispersed in the core to control odor from fluids from the user.

10. The pad claim 1 wherein the first and second layers form an edge margin on the side of the seal that is spaced apart from the core.

11. The pad of claim 1 wherein an antimicrobial and antifungal substance is dispersed in the core.

12. The pad of claim 1 wherein the pad has a bending length stiffness from about 40 mm to about 70 mm.

13. The pad of claim 1 wherein the core has an absorbency from about 20 g/mm to about 90 g/mm.

14. The pad of claim 1 wherein the super absorbent polymer in the core is from about 30% to about 70% of the total basis weight of the pad.

15. The pad of claim 1 wherein a line is positioned on at least one of the first or second layers of non-woven material, at least one fold line positioned adjacent the line, the at least one fold line providing a reference to fold the pad to produce a contour in the pad.

16. The pad of claim 16 wherein the line and the at least one fold line are printed or embossed on the at least one layer of non-woven material.

17. The pad of claim 17 wherein a plurality of fold lines are positioned on the at least one layer of non-woven material.

18. The pad of claim 16 wherein an adhesive strip is positioned on theat least one layer of non-woven material adjacent the line, the adhesive strip extending across the line and the at least one fold line.

19. The pad of claim 19 wherein the adhesive strip can be used to secure the pad in the folded position to retain the contour in the pad.
